(19) United States
(12) Patent Application Publication
(10) Pub. No.: US 2024/0148503 A1
(43) Pub. Date: May 9, 2024

(54) CATHETER SYSTEM FOR SEQUENTIAL DEPLOYMENT OF AN EXPANDABLE IMPLANT

(71) Applicant: JenaValve Technology, Inc., Irvine, CA (US)

(72) Inventors: Kevin CHU, Tustin, CA (US); Rolando LEE, Irvine, CA (US); Kendall SMITH, Costa Mesa, CA (US)

(73) Assignee: JenaValve Technology, Inc., Irvine, CA (US)

(21) Appl. No.: 18/504,932

(22) Filed: Nov. 8, 2023

Related U.S. Application Data
(60) Provisional application No. 63/383,011, filed on Nov. 9, 2022.

(51) Int. Cl. A61F 2/24; A61F 2/95

(52) U.S. Cl. CPC A61F 2/2436; A61F 2/9517

(57) ABSTRACT

Systems and methods for sequential deployment of a prosthetic valve using a delivery catheter are described. The delivery catheter may include a deployment assembly including an end cone, a distal sleeve, a prosthetic support, an anchor sleeve and an anchor support. The delivery catheter may further include a handle having a manipulator including a guide that is connect to the deployment assembly with a force transmission tube. The guide may have various channels for interfacing with a protrusion of the handle and may be guided by the protrusion to sequentially deploy the prosthetic valve from the deployment assembly. For example, the guide may cause the distal sleeve to release a distal end of the prosthetic valve and subsequently cause an anchor support to release a proximal end of the prosthetic valve.

### CROSS-REFERENCE TO RELATED APPLICATIONS

This application claims the benefit of U.S. Provisional Patent Application Ser. No. 63/383,011, filed Nov. 9, 2022, the entire contents of which are incorporated herein by reference.

### FIELD OF THE INVENTION

The present invention relates generally to catheter systems for deploying a cardiac implant. For example, systems and methods are provided herein including a catheter system for precise, sequential placement of prosthetic heart valves.

### BACKGROUND

In medical technology, there has been an endeavor to treat or fix a heart valve defect, such as an aortic valve insufficiency or an aortic valve stenosis, non-surgically using transarterial interventional access via a catheter, thus without an invasive surgical operation. Transcatheter aortic valve replacement (TAVR)/transcatheter aortic valve intervention (TAVI) procedures are becoming more commonplace. Various insertion systems and stent systems have been proposed, with different advantages and disadvantages, which in part can be introduced into the body of a patient transarterially by means of a catheter insertion system.

In the medical devices previously known, however, it has become apparent that the implantation procedure of a stent system to which the heart valve prosthesis is attached is relatively complicated, difficult, and expensive. Apart from the complicated implantation of the heart valve prosthesis as a replacement for an insufficient or defective native heart valve, there is the fundamental risk of incorrect positioning of the stent or heart valve prosthesis with the medical devices used up to the present, which cannot be corrected without more extensive and invasive surgical intervention.

It is also regarded as problematic that, when using systems already known from the state-of-the-art, incorrect positioning of the heart valve prosthesis or the associated heart valve stent can often only be avoided when the heart surgeon or interventional cardiologist is especially experienced.

Improved delivery catheter systems are described in, for example, U.S. Pat. No. 11,065,138 to Schreck, U.S. Pat. No. 11,147,669 to Straubinger, and U.S. Pat. No. 8,679,174 to Ottma, the entire contents of each of which are incorporated herein by reference.

What is needed are further improved systems and devices for introducing a sequentially expandable heart valve stent into the body of a patient, for positioning the stent at a desired implantation site, and for reducing the risk to the patient on implantation.

### SUMMARY

Provided herein are catheter systems and methods for implanting a prosthetic heart valve in a sequential manner. The catheter system may be used to implant a prosthetic heart valve having arms that allow the prosthetic heart valve to clip onto the native valve. This method of attachment allows the valve to be placed into the heart without having to sew the prosthetic valve into the heart. At this time, no delivery catheter commercially available in the U.S. is capable of performing the requisite sequential deployment for such a prosthetic valve. The delivery catheter described herein may be used to sequentially deploy the prosthetic valve. The delivery catheter may be capable of positioning the prosthetic valve over the native valve and releasing the portions of the prosthetic valve sequentially before uncoupling the valve from the delivery catheter. The delivery catheter allows for precise positioning of the prosthetic valve at a desired implantation site. The prosthetic valve can then be sequentially deployed in a controlled manner for improved precision and safety.

A catheter system for implanting a prosthetic heart valve may, in one example, include a delivery catheter including a proximal region and a distal region, the distal region sized and shaped to be advanced to an implantation site at a native heart valve and including: i) a first sleeve configured to retain a first end of the prosthetic heart valve, ii) a second sleeve configured to retain a second end of the prosthetic heart valve, and iii) a stent holder configured to engage the prosthetic heart valve. The delivery catheter may include a handle coupled to the proximal region of the delivery catheter, a manipulator configured to rotate about an axis of the manipulator and move axially with respect to the handle, and a guide coupled to the manipulator and including at least a first channel and a second channel perpendicular and connected to the first channel, the guide in mechanical communication with the stent holder and the second sleeve, the first and second channels configured to receive a protrusion as it moves within the first and second channels such that movement of the protrusion along the first channel unlocks the manipulator and movement of the protrusion along the second channel sequentially moves the second sleeve distally and stent holder distally thereby releasing the prosthetic heart valve.

The second channel may include a spring, wherein the spring permits movement of the protrusion in a first direction and resists movement in a second direction opposite the first direction. The spring may have cantilevered portion having a spring force. The cantilevered portion may be elastic. The first channel may be connected to a first end of the second channel. The guide may include a third channel connected to a second end of the second channel, wherein third channel is configured to receive the protrusion after it has moved within the second channel. The third channel may be perpendicular to the second channel.

The catheter system may further include a fourth channel perpendicular to and connected to the third channel. The fourth channel may be configured to lock the protrusion to prevent movement of the second sleeve and stent holder. The catheter system may further include a fifth channel connected to the second channel and configured to allow the protrusion to bypass the spring thereby permitting the protrusion to move in the second direction opposite the first direction. The fifth channel may include a ramp configured to guide the protrusion into the second channel.

A method for deploying a prosthetic heart valve using a catheter system including a handle and a delivery catheter coupled to the handle at a proximal end of the delivery catheter may, in one example, include advancing a distal end of the delivery catheter to an implantation site at a native heart valve, the distal end of the delivery catheter including the prosthetic valve in a collapsed, delivery state, rotating a manipulator engaged with the handle to cause a protrusion coupled to the handle to traverse a first channel of a guide disposed within the manipulator and enter a second channel of a guide perpendicular to and connected to the first channel, thereby unlocking the manipulator, the manipulator in mechanical communication with the distal end of the delivery catheter, the distal end including (i) a first sleeve fixed to the delivery catheter and including a first opening at a distal end of the first sleeve, the first sleeve configured to retain a first end of the prosthetic heart valve, (ii) a stent holder disposed within the first sleeve and axially movable with respect to the first sleeve, the stent holder configured to engage the prosthetic heart valve, and (iii) a second sleeve coupled to a tip and axially movable with respect to the first sleeve, the second sleeve including a second opening at a proximal end of the second sleeve and configured to retain a second end of the prosthetic heart valve and advancing, axially in a proximal to distal direction, the manipulator to cause the protrusion to traverse the second channel; thereby sequentially moving the second sleeve distally and the stent holder distally thereby releasing the prosthetic heart valve at the implantation site.

The method may further include advancing a sheath including a sheath marker band to a sinotubular junction in a patient's heart, inserting the delivery catheter including a sealing ring marker band into the sheath, advancing the delivery catheter though the sheath until the sealing ring marker band aligns with the sheath marker band, and aligning the distal end of the delivery catheter with the native heart valve in the patient's heart. Aligning the distal end of the delivery catheter with the native heart valve in a patient's heart may include rotating a second manipulator engaged with the handle. The method may include aligning the distal end of the delivery catheter with the native coronary cusps of the patient's heart. The method may further include removing a safety clip removeably connected to the handle before rotating the manipulator.

The method may further include rotating the manipulator engaged with the handle a second time and advancing, axially in a proximal to distal direction, the manipulator a second time. The second channel may further include a spring, wherein the protrusion moves along the second channel in a first direction past the spring, wherein the spring resists the protrusion from moving in a second direction opposite the first direction. The method may further include retracting the manipulator to cause the protrusion to move along the second channel in the second direction of the guide to a third channel connected to the second channel, thereby circumventing the spring. The protrusion may circumvent the spring by moving along a ramp in the third channel.

The method may further include rotating the manipulator to cause the protrusion to traverse the second channel in the second direction.

### BRIEF DESCRIPTION OF THE DRAWINGS

FIG. 1 depicts an exemplary delivery catheter including a deployment assembly and a handle, in accordance with some aspects of the present invention.

FIG. 2 illustrates a perspective view of an exemplary handle of the delivery catheter.

FIG. 3 illustrates a perspective view of an exemplary handle deployment assembly of the delivery catheter.

FIG. 4 illustrates a cross-sectional view of an exemplary deployment manipulator.

FIG. 5 illustrates a perspective, cross-sectional, exploded view of an exemplary deployment manipulator.

FIG. 6A illustrates a perspective view of an exemplary deployment guide within the deployment manipulator. FIG. 6B illustrates a close-up, perspective view of an exemplary portion of the deployment guide.

FIGS. 7A-7J illustrate a series of deployment configurations of an exemplary deployment guide and corresponding prosthetic heart deployment.

FIGS. 8A-8H illustrate a series of deployment configurations of an exemplary deployment guide and corresponding loading of a deployment assembly.

### DETAILED DESCRIPTION

The present invention is directed to a catheter system for introducing an expandable heart valve stent into the body of a patient. Specifically, the catheter system may include a catheter including a deployment assembly and a handle for inserting an expandable heart valve stent into the body of a patient. The expandable heart valve may be used for treatment of a heart valve defect, in particular a heart valve failure or a heart valve stenosis in a patient. The expandable heart valve stent may be accommodated in the deployment assembly on a distal end of the catheter, which may be manipulated using the handle system to sequentially deploy the expandable heart valve stent.

The delivery catheter of the present invention is particularly well-suited for sequential deployment of a prosthetic heart valve with arms that may clip onto a native valve, such as those described in U.S. Pat. No. 11,154,398 to Straubinger, the entire contents of which are incorporated herein by reference. The delivery catheter may be capable of positioning the prosthetic heart valve over the native valve, releasing one set of arms of the prosthetic heart valve thereby allowing for partial expansion of the prosthetic heart valve, and releasing a second set of arms of the prosthetic heart valve thereby allowing for further expansion of the prosthetic heart valve before uncoupling the valve from the delivery catheter for complete expansion.

For improved safety, the catheter system described herein allows for precise alignment and deployment of a prosthetic valve in the heart. The catheter system may include a delivery catheter with a manipulator and a guide in mechanical communication with a stent holder. The guide allows for sequential deployment of the prosthetic valve.

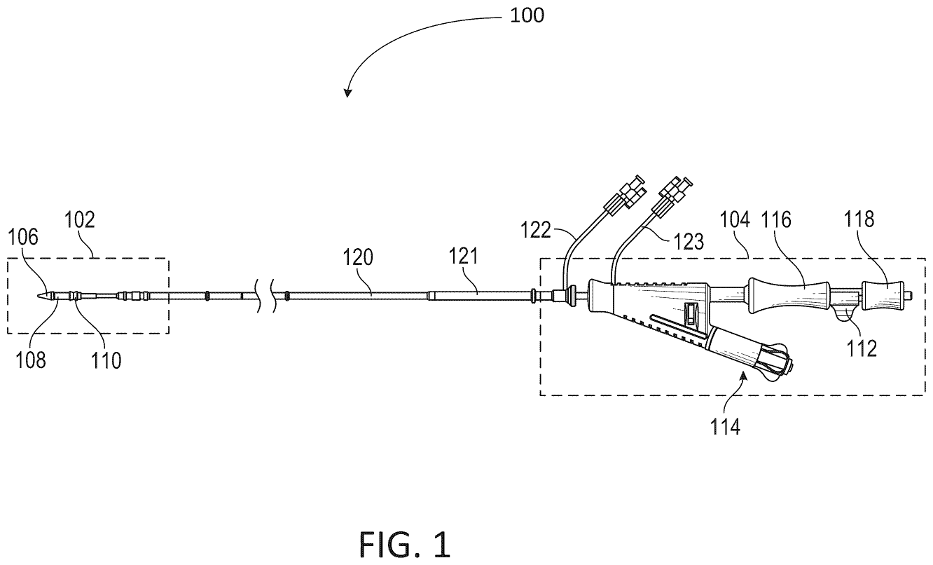

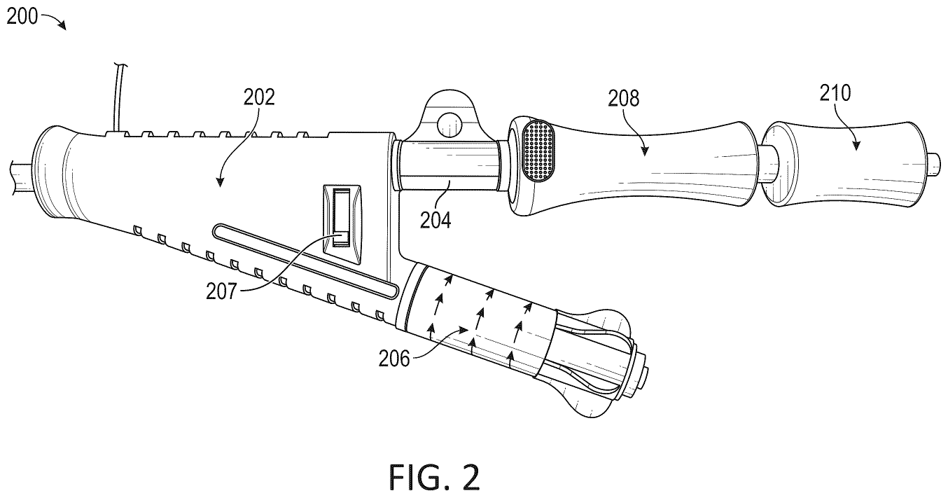

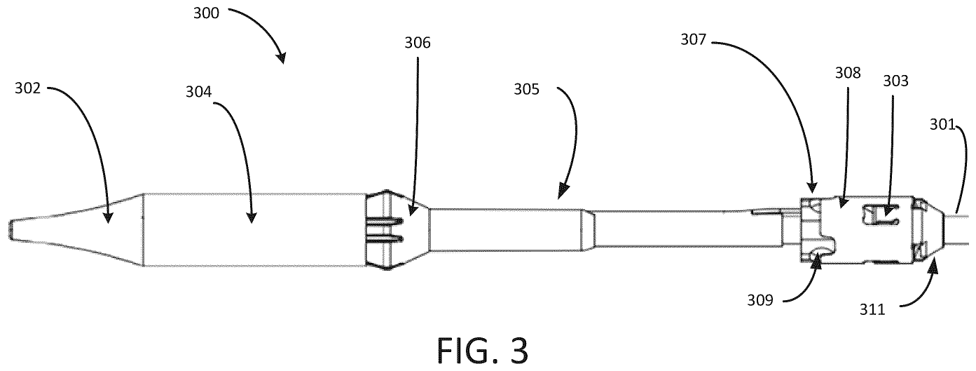

Referring now to FIG. 1, an exemplary delivery catheter including a handle system with a guide for sequential deployment of a prosthetic heart valve is illustrated. As shown in FIG. 1, delivery catheter 100 may include a proximal end and a distal end. The proximal end of delivery catheter 100 may include handle 104. Handle 104 illustrated in greater detail in FIG. 2. The distal end of delivery catheter 100 may include deployment assembly 102. Deployment assembly 102 is shown in greater detail in FIG. 3. Deployment assembly 102 and handle 104 may be operably connected via catheter portion 120. Catheter portion 120 may include a guide wire for guiding deployment assembly 102 through a blood vessel to the heart and may further include one or more force transmission tubes, wires, cables, or the like, which may translate movement of handle 104 to movement at deployment assembly 102.

Handle 104 may include bending manipulator 114, used to bend catheter portion 120, rotating manipulator 116, used to rotate the deployment assembly about a longitudinal axis of delivery catheter 100, and deployment manipulator 118 used to manipulate deployment assembly 102 to deploy a prosthetic heart valve. Bending manipulator 114 may be rotated to bend catheter portion 120 into a u-shape to navigate the vasculature of the body (e.g., the aortic arch). In one example, a cable or other force transmitter may extend from bending manipulator 114 to deployment assembly 102 or a distal end of catheter portion 120 and when bending manipulator 114 is rotated, the cable or force transmitter may shorten in length causing the distal end of delivery catheter 100 to bend towards handle 104.

Rotating manipulator 116 may be a handle that may be rotated about a longitudinal axis of handle 104 to cause deployment assembly 102 to similarly rotate about a longitudinal axis of delivery catheter 100. For example, rotating manipulator 116 may be in mechanical communication with deployment assembly 102 (e.g., via a force transmission tube) and as rotating manipulator 116 is rotated, deployment assembly 102 may similarly rotate (e.g., clockwise or counter-clockwise).

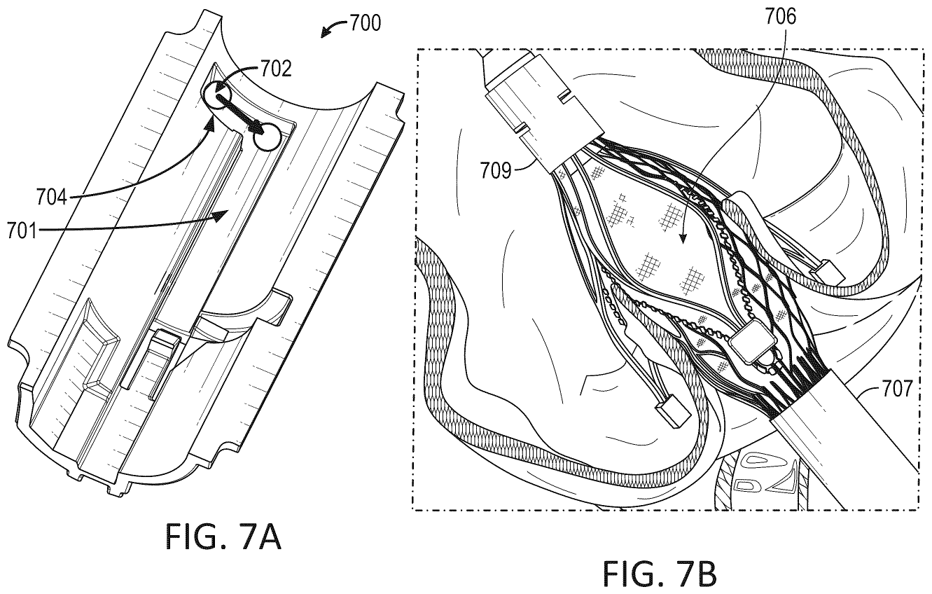

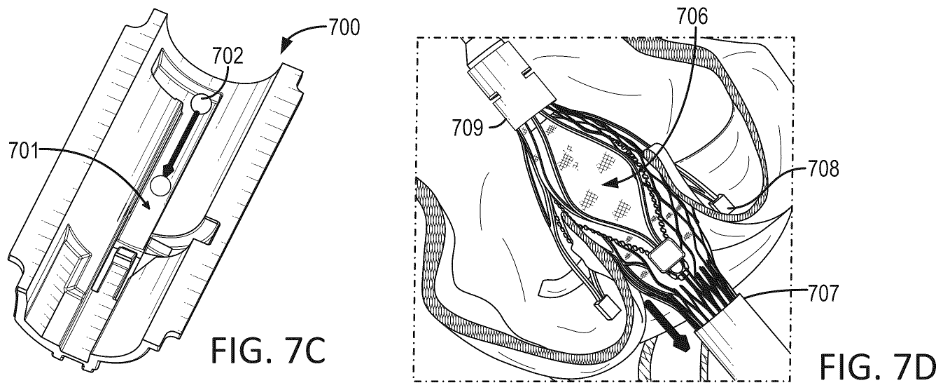

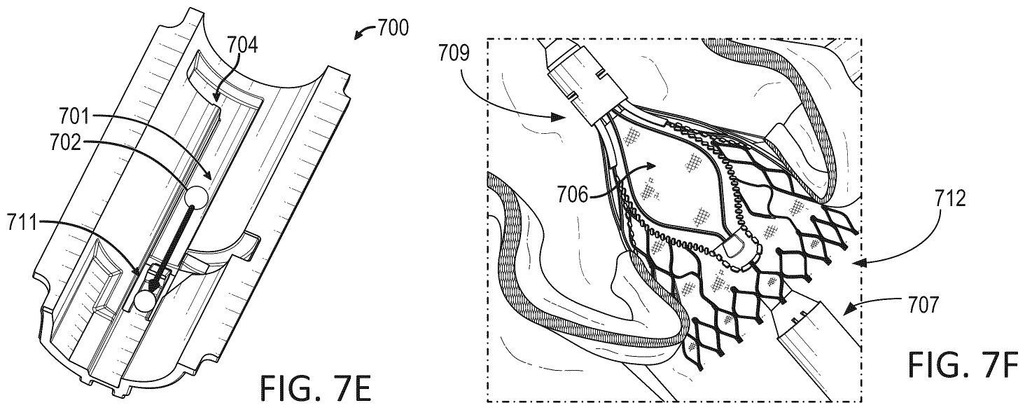

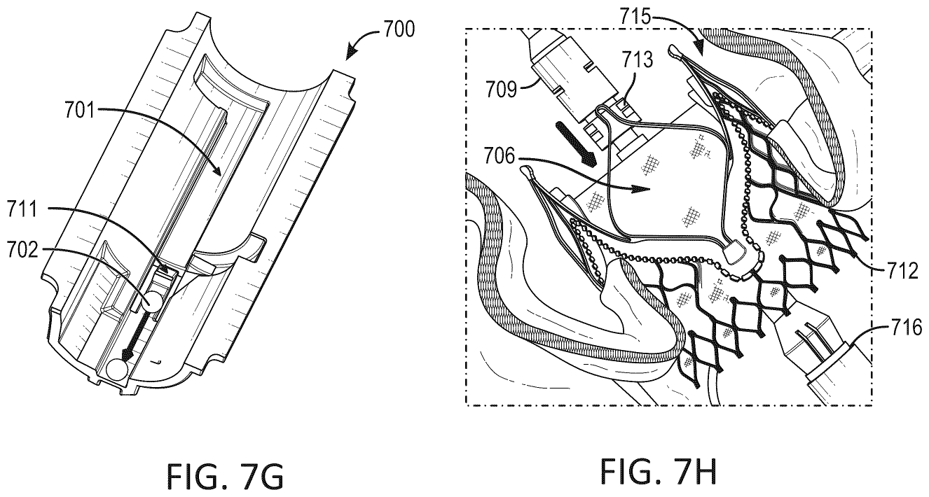

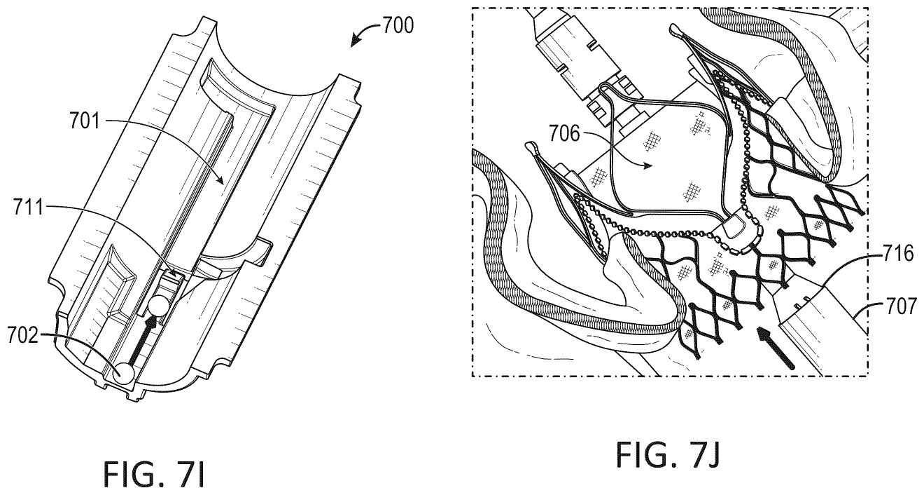

Deployment manipulator 118 may include a guide that causes sequential manipulation of deployment assembly 102. Deployment assembly 102 may include end cone 106, distal sleeve 108, and prosthetic holder 110. As deployment manipulator 118 is manipulated (e.g., as described below with respect to FIGS. 7A-7J), distal sleeve 108 and a stent holder on prosthetic holder 110 may be manipulated in sequence to sequentially expand and release a prosthetic heart valve positioned on deployment assembly 102.

Handle 104 may further include safety clip 112 which may be removable and may be used to prevent deployment manipulator 118 from advancing, thereby preventing the deployment assembly 102 from releasing the prosthetic heart valve. Further, delivery catheter 100 may include port 122 and port 123 for permitting flushing of internal channels within catheter portion 120 and/or handle 104. Additionally, delivery catheter 100 may include introducer sheath 121 which may interface with handle 104 and in which catheter portion 120 may be positioned. In one example, port 122 may be used to flush introducer sheath 121.

To deliver the prosthetic heart valve to the implantation site, a sheath may first be placed in a patient's blood vessel and pushed through the patient's body until the sheath reaches the heart. Delivery catheter 100 may be advanced through the sheath until it reaches the heart. Once deployment assembly 102 is at the patient's heart, the sheath may be fully or partially retracted. In one example, the sheath may be retracted before delivery catheter 100 is removed from the patient's body.

Delivery catheter 100 may be threaded through vasculature towards the heart and may be bent to conform to the shape of the vasculature. For example, delivery catheter 100 may be introduced to an artery in the patient's leg, such as the transfemoral artery. In embodiments, delivery catheter 100 may be placed in the transfemoral artery in a patient's leg. Delivery catheter 100 may then be pushed through the transfemoral artery until it reaches the heart. It is understood that such an approach may include pushing delivery catheter 100 through the leg following the transfemoral artery until it curves around the top of the heart to reach the aorta; accordingly, when deployment assembly 102 reaches the aorta, it may have turned at least 90 degrees and be facing approximately the opposite direction as it had when it first entered the patient's body through the patient's leg. In such cases, bending manipulator 114 may cause deployment assembly 102 to move in an arch.

Upon reaching the native valve (e.g., aortic valve), rotating manipulator 116 may be rotated about its axis. Specifically, when rotating manipulator 116 is rotated, deployment assembly 102 may rotate in the same direction. Rotating manipulator 116 may allow for more controlled and more precise angular movement to align the prosthetic valve with the anatomy of the native valve. In one example, rotating manipulator 116 may have a button to allow for additional control and enhanced safety. For example, rotating manipulator 116 may be locked and restrained from moving.

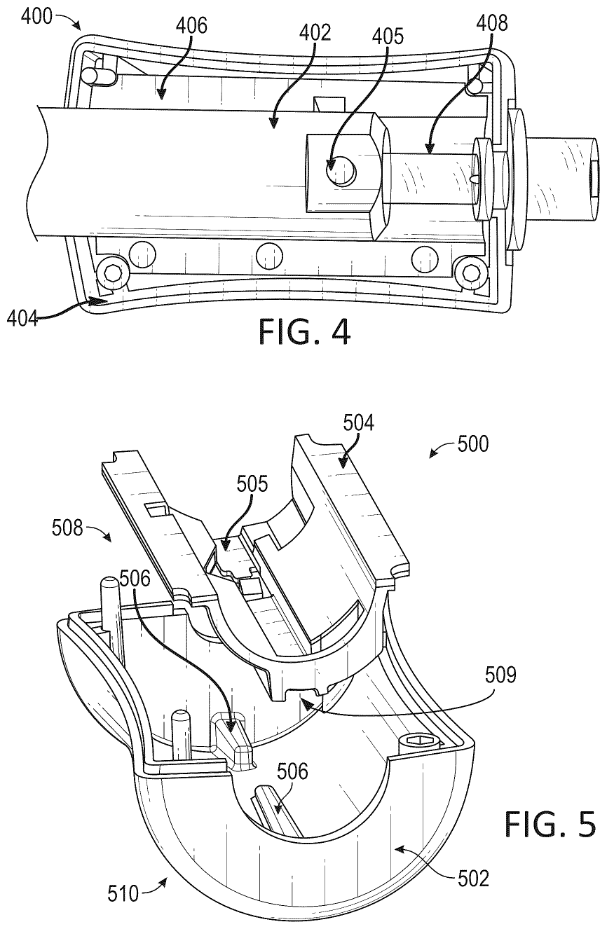

Deployment manipulator 118 is configured to rotate about its axis and move axially with respect to handle 104. For example, a user may alternate between rotating and advancing deployment manipulator 118 to sequentially deploy the prosthetic valve. Deployment manipulator 118 may be configured to release the prosthetic valve in sequential steps using a guide. The deployment manipulator 118 and the guide are described in greater detail in FIGS. 4-5.

It is understood the delivery catheter 100 may be used to implant a prosthetic heart valve and/or may be used to implant other prosthetic devices, such as stents. For example, delivery catheter 100 may be used to sequentially deploy a prosthetic heart valve that has a set of position arches and a set of retaining arches. A user such as a surgeon or cardiologist may hold handle 104 and manipulate handle 104 to navigate the cardiovascular system of the patient and ultimately sequentially expand the prosthetic heart valve to cause the prosthetic heart valve to clip onto the heart leaflets to anchor the prosthetic heart valve to a native valve. In this manner such a prosthetic heart valve may be implanted without the need be sewn to the heart.

Referring now to FIG. 2, handle 200, which may be the same as or similar to handle 104 described above with respect to FIG. 1 is illustrated. Handle 200 may include handle support 202, which may be and/or include a handle housing that may be used by the user to grip handle 200 and/or any other handle portion. Handle support 202 may be connected to bending manipulator 206, rotating manipulator 208, and deployment manipulator 210. Bending manipulator 206 may be the same or similar to bending manipulator 114. Rotating manipulator 208 may be the same or similar to rotating manipulator 116. Deployment manipulator 210 may be the same or similar to deployment manipulator 118.

As shown in FIG. 2, handle 200 may further include safety clip 204, which may be positioned between rotating manipulator 208, handle support 202, and/or deployment manipulator 210. For example, safety clip may be located between handle support 202 and rotating manipulator 208, but in other examples, safety clip 204 may be located between rotating manipulator 208 and deployment manipulator 210 instead. Safety clip 204 may obstruct movement of rotating manipulator 208 and/or deployment manipulator 210 and may need to be removed before deployment can begin. Handle 200 may further include safety lock 207 which may be a switch that may lock movement bending manipulator 206 in position and may prevent movement of bending manipulator 206 in another position.

Safety clip 204, bending manipulator 206, rotating manipulator 208, and deployment manipulator 210 may be shaped and sized to be grasped and easily manipulated by hand. For example, safety clip 204, bending manipulator 206, rotating manipulator 208, and deployment manipulator 210, may have indentations, raised patterns, and/or groves to improve a user's grip. Safety clip 204 may be shaped to allow a user to easily pinch safety clip 204 between two fingers. Bending manipulator 206, rotating manipulator 208, and deployment manipulator 210 may have a smaller diameter at their centers than their ends to improve a user's grip.

Referring now to FIG. 3, deployment assembly 300 is illustrated in greater detail. Deployment assembly 300 may include end cone 302, distal sleeve 304, taper assembly 306, prosthetic support 305, prosthetic holder 307, anchor support 309, anchor sleeve 308, cone 311 and catheter tube 301. End cone 302 may be connected to distal sleeve 304. For example, end cone 302 and distal sleeve 304 may be one continuous component or may be separate components connected or joined with one another. End cone 302 and/or distal sleeve 304 may be made from a plastic material have an elastic or compressible structure, for example. Distal sleeve 304 may be open at a proximal end. End cone 302 and/or distal sleeve 304 may be connected to anchor sleeve 308 and may cause anchor sleeve 308 to move when end cone 302 and/or distal sleeve 304 reach a certain position.

Prosthetic support 305 and taper assembly 306 may be coupled together. It is understood that taper assembly 306 may include several slits and/or may be made of elastic material and may be compressible such that distal sleeve 304 may traverse an apex of taper assembly 306. A prosthetic device, such as a prosthetic heart valve may be positioned upon prosthetic support 305 and/or may abut or otherwise be supported by taper assembly 306. Prosthetic support 305 may be tubular in shape and may be coupled to anchor support 309. Anchor support 309 may be coupled to cone 311 and which may be coupled to catheter tube 301.

Anchor support 309 is sized and shaped to receive or otherwise engage a portion of the prosthetic device positioned upon prosthetic support 305. Anchor sleeve 308 may be a tubular sleeve that may extend over anchor support 309 such that anchor sleeve may completely cover anchor support 309, thereby securing the engaged portion of the prosthetic device into anchor support 309. Anchor sleeve 308 may optionally include locking structure 303 that, when anchor sleeve 309 is positioned over anchor support 309, locking structure 303 may secure anchor sleeve 309 into place. In one example, locking structure 303 may be released by an exterior tube (not shown) that may be positioned over anchor sleeve 308. Cone 311 may provide a gradual transition from the diameter of anchor sleeve 308 to the diameter of catheter 301, prevent damage to the vasculature of the patient during implantation of the prosthetic device.

Referring now to FIG. 4, a cross-sectional view of deployment manipulator 400 is illustrated. Deployment manipulator 400 may be the same as or similar to deployment manipulator 118. Deployment manipulator 400 may be connected to handle support 402, which may connect deployment manipulator 400 to the rest of a handle (e.g., handle 104 of FIG. 1). Handle support 402 may be tubular in shape and/or may be or include one or more handle portions. Handle support 402 may include protrusion 405. Protrusion 405 may be positioned at a distal end of handle support 402 and/or at any other position of handle support 402. Protrusion 405 and/or any other protrusion described herein may be any type of protrusion or pin that may extend outwardly from a surface of handle support 402. Deployment manipulator 400 may be designed to accommodate handle support 402. Deployment manipulator 400 may be rigidly attached to force transmission tube 408 which may extend through the handle and a catheter and connect to a deployment assembly (e.g., deployment assembly 102 of FIG. 1). It is understood that force transmission tube 408 may be any well-known force transmission structure (e.g., cable, tube, rod, etc.). Force transmission tube 408 may be coupled to housing 404 of deployment manipulator 400 such that axially movement of deployment manipulator 400 may be translated to force transmission tube 408, but rotational movement may not.

Axial movement of force transmission tube 408 may cause a distal sleeve (e.g., distal sleeve 304 of FIG. 3) and an anchor sleeve (e.g., anchor sleeve 308) of a deployment assembly to move sequentially. Deployment manipulator 400 may include housing 404 which may contain guide 406. Guide 406 may receive the distal end of handle support 402 which may be halved into two parts and include a series of channels that receive protrusion 405 and deployment manipulator 400 to move axially with respect to protrusion 405 and handle support 402.

FIG. 5, is an exploded cross-sectional view of deployment manipulator 500. Deployment manipulator 500 may be the same or similar to deployment manipulator 400. As shown in FIG. 5, only half of deployment manipulator 500 is illustrated. It is understood that a similar half (not shown) may be connected to the components illustrated in FIG. 5 to form the complete deployment manipulator 500, however the half not shown may not have channels 505. It is understood that the term channel used throughout may be any channel and/or a guiding structure, for example. It is further understood that the channel may be one or more recessed portions and/or may protrude outward from a surface. Deployment manipulator 500 may include housing 502 which may be a cylindrical shell and may further include guide 504. Housing 502 may be configured to be easily held and manipulated by a user and may have a center portion having a smaller diameter than a proximal and distal end.

Guide 504 may be designed to be positioned within and secured to housing 502. Housing 502 may further be secured to a force transmission tube (e.g., force transmission tube 408 of FIG. 4). For example, housing 502 may include grooves 506 that protrude inward and guide 504 may include groove receiving portions 509 sized to receive grooves 506 to hold guide 504 within housing 502. Guide 504 may further be secured to housing 502 via any other well-known securing techniques (e.g., glue, threaded engagement, welding, etc.).

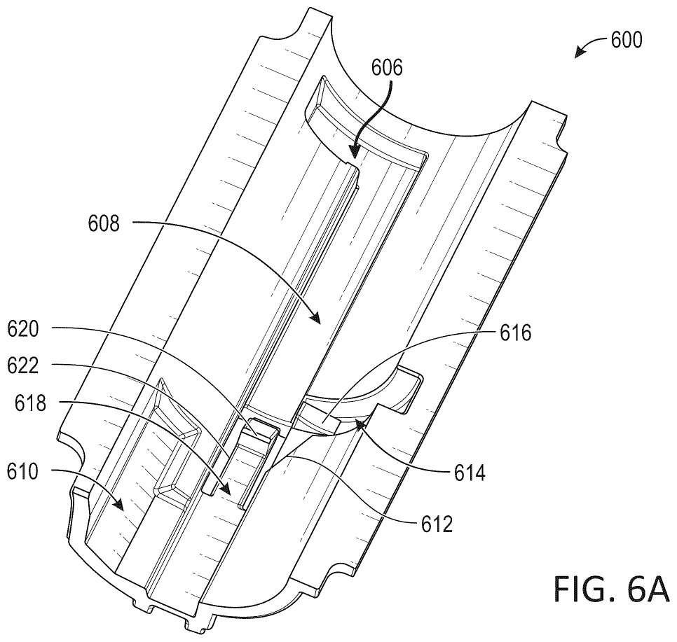

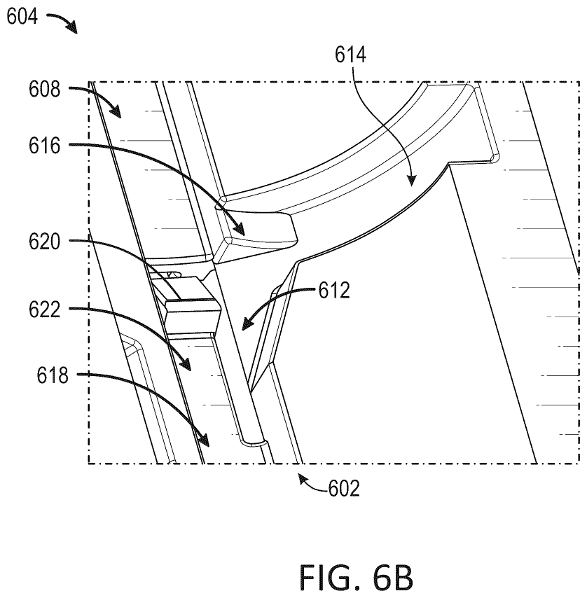

Deployment manipulator 500 has proximal end 508 and distal end 510. Proximal end 508 may receive a handle support (e.g., handle support of FIG. 402). Distal end 510 may be sized and shaped to engage a force transmission tube (e.g., force transmission tube 408 of FIG. 4). Guide 504 may further include channel 505 which may be a channel sized to receive and guide a protrusion (e.g., protrusion 402 of FIG. 4) along channel 505. FIGS. 6-8 show in greater detail how movement of a protrusion along guide 504 allows for sequential deployment of a prosthetic valve.

Referring now to FIGS. 6A-6B, a perspective view of guide 600 and focused view of spring 618 is illustrated. Guide 600 may be the same or similar to guide 504 of FIG. 5. As shown in FIG. 5, guide 600 may include various protrusion guiding channels, which may be recessed grooves within guide 600 for receiving a protrusion, for example. In one example, guide 600 may include locking channel 606, delivery channel 608 including spring 618, storage channel 610, bypass channel 612, and ramp channel 614 including ramp 616. The various channels may be sized to receive a protrusion and allow the protrusion to traverse the channels. In this manner, the deployment manipulator may guide the protrusion to move along locking channel 606 to delivery channel 608 resulting in sequential deployment of a prosthetic valve.

The protrusion may start in locking channel 600. Locking channel 600 may provide a space for the protrusion to be stationary before deployment. In this position, a prosthetic valve may be retained by the delivery catheter while the protrusion is in locking channel 606. To prepare the deployment manipulator for deployment of the prosthetic valve, a user must rotate the deployment manipulator to cause the protrusion to traverse locking channel 606 and guide the protrusion into delivery channel 608.

Locking channel 606 may be perpendicular to and connected to delivery channel 608. Locking channel 606 may be orthogonal to a longitudinal axis of the deployment manipulator and delivery channel 608 may be parallel to the longitudinal axis of the deployment manipulator. In this manner, delivery channel 608 may be perpendicular to locking channel 606. The protrusion may move from locking channel 606 into delivery channel 608 as the deployment manipulator is rotated about its longitudinal axis.

A protrusion may be caused to traverse delivery channel 608 as the deployment manipulator is advanced with respect to the handle. Delivery channel 608 may include spring 618 along delivery channel 608. Spring 618 may include cantilevered portion 622 that may include an angled obstruction 620 at a distal end that permits movement in only one direction along delivery channel 608 but resists movement of the protrusion in the opposite direction. For example, a protrusion may engage angled obstruction 620 such that advancement of the protrusion along delivery channel 608 causes a downward force on cantilevered portion 622 of spring 618 thereby causing cantilevered portion 622 of spring 618 to deflect downward permitting the protrusion to traverses spring 618 and delivery channel 608. It is understood that cantilevered portion 622 may have a spring force that permits the cantilevered portion 622 to deflect elastically.

Upon traversing spring 618, the protrusion may be unable to move back towards locking channel 606 as angled obstruction 620 restricts movement in the opposite direction. For example, angled obstruction 620 may be shaped such that movement in the opposite direction is not translated to a downward force on the cantilevered portion. Accordingly, the protrusion may only be able to move in one direction along spring 618. Such restricted movement assists with safety and precision, as the spring force may be strategically positioned along delivery channel 608 to provide tactile feedback to indicate certain movement of the deployment assembly has been achieved. For example, spring 618 may be strategically positioned along delivery channel 608 to indicate that the distal sleeve has released a distal portion of the prosthetic valve.

In addition to locking channel 606, Delivery channel 608 may be connected to storing channel 610 which may include a portion perpendicular to delivery channel 608 and a portion parallel to delivery channel 608. Storing channel 610 is configured to receive the protrusion while the delivery catheter is in storage, transit or otherwise not in use. Storing channel 610 allows the protrusion to be stored with guide 600 in a neutral position prior to loading the prosthetic valve into the deployment assembly.

To load the prosthetic valve, the deployment manipulator may be advanced and subsequently rotated to guide the protrusion out of storing channel 610 and into delivery channel 608. Upon entering delivery channel 608, the deployment manipulator must be retracted with the respect to the handle to guide the protrusion along delivery channel 608 towards locking channel 606. As protrusion advances along delivery 608 towards locking channel 606, protrusion may be obstructed by spring 618.

Referring now to FIG. 6B a close-up view of guide 600 including spring 618, and bypass channel 612 in greater detail. As the protrusion is caused to be advanced along delivery channel 608 toward locking channel 606, spring 618 may be circumvented by use of bypass channel 612. As shown in FIG. 6B, bypass channel by extend diagonally form delivery channel 608 and offer a diverted path for protrusion around spring 618. For example, a user may feel the tactile response from spring 618 and in response rotate the deployment manipulator to cause the protrusion to enter bypass channel 612.

Upon entering bypass channel 612, the protrusion may then be guided in ramp channel 614, which may be perpendicular to delivery channel 608. Ramp channel 614 may include ramp 616 which may be flush with ramp channel 614 at one end and may extend upward toward delivery channel 608, such that ramp 616 terminates at a height taller than a floor of delivery channel 608. In this manner, ramp 616 may serve as a wall for delivery channel 608 and prevent the protrusion from entering ramp channel 614 as it traverses delivery channel 608 towards spring 618.

Bypass channel 612 may thus permit the protrusion to bypass spring 618 and guide the protrusion into ramp channel 614. Once the protrusion enters ramp channel 614, a user may feel resistance to further advancement of the deployment manipulator and in response may rotate deployment manipulator to cause the protrusion to climb ramp 616 and ultimately reenter delivery channel 608, thereby bypassing spring 618 including angled obstruction 620. Upon reentering delivery channel 608, the protrusion may traverse delivery channel 608 towards locking channel 606.

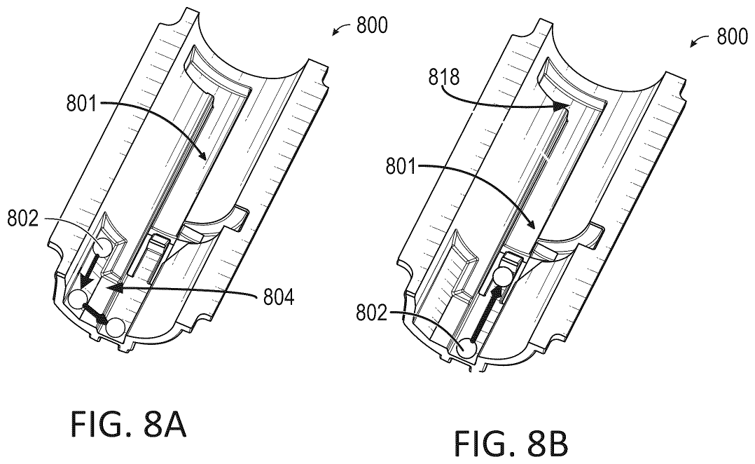

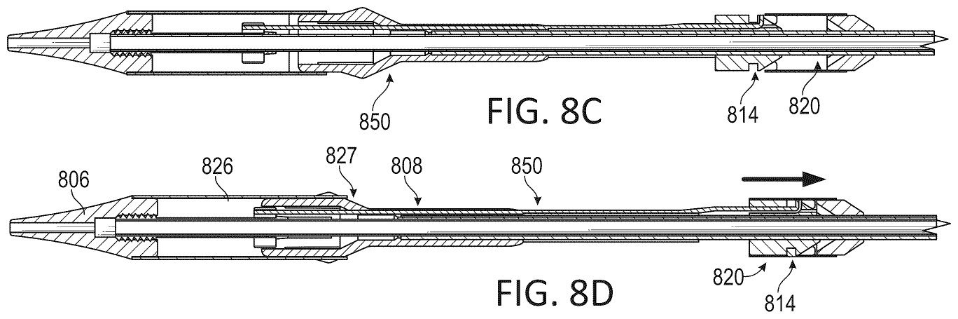

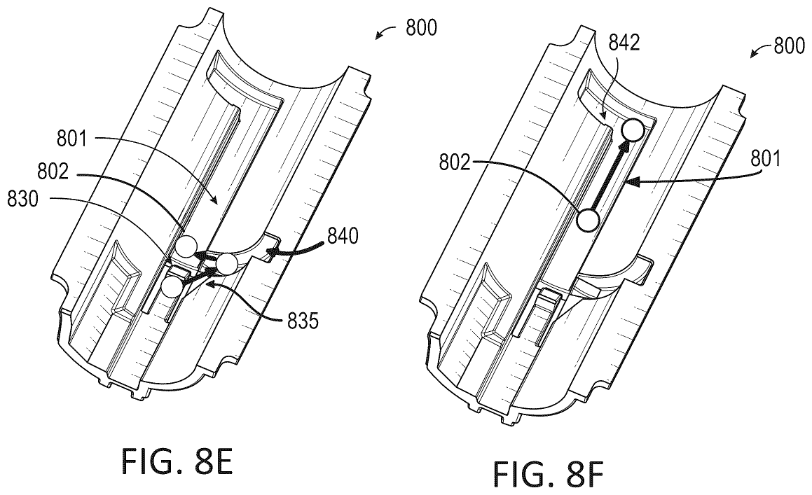

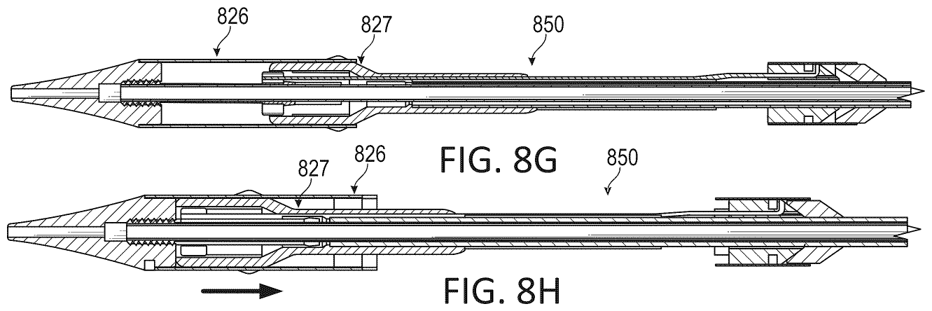

Manipulating the deployment manipulator and thus guide 600 with respect to the protrusion to deploy the prosthetic valve, is described in more detail in FIGS. 7A-7J. Manipulating the deployment manipulator and thus guide 600 with respect to the protrusion to load the prosthetic valve to into the deployment assembly of the delivery catheter is described in more detail in FIGS. 8A-8H.

Referring now to FIGS. 7A-7J, deployment of a prosthetic heart valve using a guide is illustrated. As shown in FIGS. 7A, protrusion 702, which may be the same as or similar to protrusion 405 of FIG. 4, may begin in locking channel 704 of guide 700. Guide 700 may be the same as or similar to guide 600 and locking channel 704 may be the same as or similar to locking channel 606 of FIG. 6. Referring now to FIG. 7B, when protrusion 702 is in the locked position illustrated in FIG. 7A, prosthetic valve 706 may be fully retained by deployment assembly 705, which may be the same as or similar to deployment assembly 300 of FIG. 3. Deployment assembly 705 may include distal sleeve 707, which may be the same as distal sleeve 304 of FIG. 3, and anchor sleeve 709, which may be the same as anchor sleeve 308 of FIG. 3. As shown in FIG. 7B, prosthetic valve 706 may be retained at a proximal end by anchor sleeve 709 and an anchor support (not shown) and may be retained at a proximal end by distal sleeve 707 at a distal end. In this manner, when 702 is in locking channel 704, prosthetic valve 706 may be fully compressed.

The position of protrusion 702 may be ideal for advancing prosthetic valve 706 through the patient's vasculature and into the patient's heart. Once prosthetic valve 706 is properly aligned within the patient's heart, protrusion 702 may be advanced towards delivery channel 701. For example, a user may rotate deployment manipulator to cause guide 700 to similarly rotate. Deployment may also be impeded by a safety clip thus must be removed. Once the safety clip is removed, protrusion 702 may be is able to move into delivery channel 701.

Referring now to FIGS. 7C-7D, a user may initiate deployment of prosthetic valve 706 by advancing protrusion 702 down delivery channel 701. A user is able to cause protrusion 702 to traverse guide 700 using the deployment manipulator housing guide 700, by pushing/pulling the deployment manipulator to cause protrusion 702 to move with respect to delivery channel 701 away from locking channel 704. As shown in FIG. 7D, advancing protrusion 702 along delivery channel 701 causes distal sleeve 707 to move distally while anchor sleeve 709 remains in place.

Referring now to FIGS. 7E-7F, as protrusion 702 is caused to continue to traverse delivery channel 701, protrusion 702 will engage with, deform, and advance beyond spring 711, which may be the same as spring 618 of FIG. 6. Such manipulation provides tactile input to the user during deployment in that traversing spring 711 will be met with initial resistance and then release. Spring 711 may be strategically placed along delivery channel 701 such that traversing spring 711 coincides with distal sleeve 707 full releasing distal end of prosthetic device 706, thereby permitting distal end 712 to expand, as shown in FIG. 7F. As also shown in FIG. 7F, anchor sleeve 709 remains in place without moving while distal sleeve 707 fully releases distal end 712 of prosthetic valve 706.

Referring now to FIGS. 7G-7H, full release of prosthetic valve 706 using guide 700 is illustrated. As shown FIG. 7G, protrusion 702 may be caused, using deployment manipulator, to continue to traverse delivery channel 701. As protrusion 702 traverses spring 711, deployment manipulator may cause anchor support 713 to move distally, thereby fully exposing supports and permitting a proximal end of prosthetic device 706 to fully expand, thereby fully releasing prosthetic valve 706 from the deployment assembly. In this manner, guide 700 may facilitate sequential deployment of prosthetic valve, releasing distal end 712 first and then proximal end 715 prosthetic valve 706.

Referring now FIGS. 7G-7H, upon fully releasing prosthetic valve 706 using guide 700, the deployment manipulator may be cause to move protrusion 702 backwards along delivery channel 701 (i.e., towards locking channel 704) and in doing so, deployment manipulator may cause distal sleeve 707 to move proximally and engage taper assembly 716, which may be the same or similar to taper assembly 306. Taper assembly 716 may be compressed by distal sleeve 707 and thus taper assembly 716 may begin to enter distal sleeve 707.

The engagement of distal sleeve 707 and taper assembly 716 may be such taper assembly 716 may prevent damage to the vasculature or other tissue of the patient from the open end of distal sleeve 707. Further, referring again to FIG. 71, the position of spring 711 along delivery channel 701 may be such that movement of protrusion 702 along delivery channel 701 may be impeded by spring 711 when distal sleeve 707 has achieved desired placement over taper assembly 716.

Referring now to FIGS. 8A-8H, release of a prosthetic valve using a guide within a deployment manipulator is illustrated. As shown in FIG. 8A, protrusion 802, which may be the same as protrusion 702 of FIGS. 7A-7J, may traverse guide 800, which may be the same as guide 700 of FIGS. 7A-7J. Guide 800 may include storing channel 804, which may be the same as storing channel 610, may permit deployment assembly to remain in a stored position (e.g., for transit or storage). The delivery catheter may be transitioned for prosthetic valve loading by causing protrusion 802 to traverse storing channel 804 and enter delivery channel 801, as is illustrated in FIG. 8A. Referring now to FIG. 8B, to prepare the deployment assembly for receiving the prosthetic valve, deployment manipulator may be pushed/pulled to cause protrusion 802 to traverse delivery channel 801 towards locking channel 818.

Referring now to FIGS. 8C-8D, deployment assembly 850 is illustrated and corresponds to protrusion 802 movement of FIGS. 8A-8B. Deployment assembly 850 may be the same or similar to deployment assembly 300 illustrated in FIG. 3. Deployment assembly 850 may include end cone 806, distal sleeve 826, taper assembly 827, prosthetic support 808, anchor sleeve 820, and anchor support 814, which may be the same or similar to end cone 302, distal sleeve 304, taper assembly 306, prosthetic support 305, anchor sleeve 308 and anchor support 309, respectively.

In moving deployment manipulator to achieve movement of protrusion 802, anchor support 814 may move in the distal direction and subsequently distal sleeve 826 may also move in the distal direction as illustrated in FIGS. 8C-8D. When anchor support 814 moves distally, prosthetic valve's proximal end, which may include anchors, may be placed into anchor support 814. As protrusion 802 traverses delivery channel 801, anchor support 814 may move proximally into anchor sleeve 820 and thus the proximal end of the prosthetic valve may be secured to deployment assembly 850.

Referring now to FIGS. 8E-8H, movement of guide 800 and deployment assembly 850 for securing the distal end of the prosthetic device is illustrated. As shown in FIG. 8E, upon manipulating guide 800 such that protrusion 802 engages spring 830, a user may receive tactile feedback from spring 830 and in response rotate and advance guide 800 such that protrusion 802 enters bypass channel 835 and subsequently ramp channel 840. A user may then rotate guide 800 such that protrusion 802 is caused to exit ramp channel 840 (e.g., a ramp the same as or similar to ramp 616 of FIG. 6B) and enter delivery channel 801. In bypassing spring 830, the distal sleeve of the deployment assembly may be permitted to advance over the taper assembly. At FIG. 8F, guide 800 may be pushed/pulled to cause (e.g., by pushing or pulling a deployment manipulator) to cause the distal sleeve to close and capture the distal end of the prosthetic valve, thereby securing the prosthetic heart valve to deployment assembly 850 in a collapsed position.

Referring now to FIGS. 8G-8H, deployment assembly 850 is illustrated with distal sleeve 826 moving proximally. FIG. 8G corresponds to FIG. 8E and illustrates that as protrusion 802 of FIG. 8E traverses bypass channel 835 and ramp channel 840 to bypass spring 830 of FIG. 8E, distal sleeve 826 is advanced over taper assembly 827. FIG. 8F illustrates that as protrusion 802 of FIG. 8E traverses delivery channel 801 towards locking channel 842, distal sleeve 826 in corresponding FIG. 8H continues to move proximally with respect to taper assembly 827. As the prosthetic valve is secured to the proximal end of deployment assembly 850, the distal end of the prosthetic valve may be contracted and surrounded by distal sleeve 826 as it moves proximally to fully secure the prosthetic valve into deployment assembly 850 in a collapsed state. To maintain the prosthetic valve in the delivery channel 801 in the collapsed state, guide 800 may be rotated to cause protrusion 802 of FIG. 8F to enter locking channel 842.

The foregoing description of illustrative embodiments has been presented for purposes of illustration and of description. It will of course be understood that the embodiments described herein are illustrative, and components may be arranged, substituted, combined, and designed in a wide variety of different configurations, all of which are contemplated and fall within the scope of this disclosure. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A delivery catheter for implanting a prosthetic heart valve, the delivery catheter comprising:
a deployment assembly disposed at a distal end of the delivery catheter, the deployment assembly sized and shaped to be advanced to an implantation site at a native heart valve and comprising a sleeve configured to retain at least a portion of the prosthetic heart valve in a collapsed, delivery state;
a handle portion disposed at a proximal end of the delivery catheter and comprising a protrusion; and
a guide in mechanical communication with and configured to rotate with respect to the handle portion and comprising a first channel configured to receive the protrusion, the guide configured to translate movement of the protrusion along the first channel to the sleeve to cause distal movement of the sleeve to release the prosthetic heart valve.

2. The delivery catheter of claim 1, wherein the first channel comprises a spring that permits movement of the protrusion in a first direction along the first channel and resists movement of the protrusion in a second direction opposite the first direction, and wherein the spring is configured to provide tactile feedback upon the sleeve releasing the prosthetic heart valve.

3. The delivery catheter of claim 2, wherein the guide further comprises a bypass channel connected to the first channel and configured to allow the protrusion to bypass the spring thereby permitting the protrusion to move in the second direction opposite the first direction.

4. The delivery catheter of claim 3, wherein bypass channel comprises a ramp configured to guide the protrusion into the first channel from the bypass channel.

5. The delivery catheter of claim 2, wherein the spring is a cantilevered portion of the first channel having a spring force.

6. The delivery catheter of claim 5, wherein the cantilevered portion is elastic.

7. The delivery catheter of claim 1, wherein the guide further comprises a second channel connected to a first end of the first channel, the second channel configured to lock the protrusion when the prosthetic heart valve is loaded into the deployment assembly.

8. The delivery catheter of claim 7, wherein the second channel is perpendicular to the first channel.

9. The delivery catheter of claim 7, wherein the guide further comprises a third channel connected to a second end of the first channel, the third channel perpendicular to the first channel.

10. The delivery catheter of claim 9, further comprising a fourth channel perpendicular to and connected to the third channel.

11. The delivery catheter of claim 10, wherein the fourth channel is configured to lock the protrusion when the deployment assembly is unloaded.

12. A method for deploying a prosthetic heart valve using a delivery catheter, the method comprising:
advancing a deployment assembly disposed at a distal end of the delivery catheter to an implantation site at a native heart valve, the deployment assembly comprising a sleeve adapted to retain at least a portion of the prosthetic valve in a collapsed, delivery state; and
advancing a guide comprising a first channel, in mechanical communication with the sleeve, and engaged with a handle portion disposed at a proximal end of the delivery catheter, the handle portion comprising a protrusion, to cause the protrusion to traverse the first channel, thereby causing the sleeve to move distally to release the at least a portion of the prosthetic heart valve.

13. The method of claim 12, further comprising:
advancing a sheath comprising a sheath marker band to a sinotubular junction in a patient's heart,
inserting the delivery catheter comprising a sealing ring marker band into the sheath;
advancing the delivery catheter though the sheath until the sealing ring marker band aligns with the sheath marker band; and
aligning the distal end of the delivery catheter with the native heart valve in the patient's heart.

14. The method of claim 13, where aligning the distal end of the delivery catheter with the native heart valve in a patient's heart comprises rotating a manipulator engaged with the handle portion.

15. The method of claim 12, further comprising removing a safety clip removeably connected to the handle portion before advancing the guide.

16. The method of claim 12, further comprising:
rotating the guide engaged with the handle portion, prior to advancing the guide, to cause the protrusion to traverse a second channel perpendicular to and connected to the first channel, the second channel adapted to lock the protrusion when the prosthetic heart valve is loaded into the deployment assembly.

17. The method of claim 12, wherein the first channel comprises a spring that resists the protrusion from moving in a second direction opposite the first direction, and wherein the spring is positioned within the first channel to cause tactile feedback upon the sleeve releasing the at least a portion of the prosthetic heart valve.

18. The method of claim 17, further comprising:
retracting the guide to cause the protrusion to move along the first channel in the second direction to a bypass channel connected to the second channel, thereby circumventing the spring.

19. The method of claim 18, wherein the protrusion circumvents the spring by moving along a ramp in the bypass channel.

20. The method of claim 18, further comprising:
rotating the guide to cause the protrusion to traverse the second channel in the second direction.
